(19) United States
(12) Patent Application Publication
(10) Pub. No.: US 2007/0087417 A1
(43) Pub. Date: Apr. 19, 2007

(54) Multiplex polynucleotide synthesis

(76) Inventor: Eugeni Namsaraev, Sunnyvale, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/250,986

(22) Filed: Oct. 14, 2005

(51) Int. Cl. C12P 19/34; C12N 15/00

(52) U.S. Cl. 435/91.1; 435/440

(57) ABSTRACT

The invention provides a method of convergently synthesizing mixtures of either single stranded or double stranded polynucleotides. In one aspect, oligonucleotides that form components of such polynucleotides are synthesized on one or more microarrays, or other large-scale parallel solid phase synthesis platforms, after which they are amplified directly, or are released into solution and then amplified. At least two sets of such released and amplified oligonucleotides are produced, referred to herein as first and second amplicons. The first and second amplicons are cleaved and then ligated to different ends of a bridging duplex that is present in the reaction in limiting quantity to form a polynucleotide mixture of the invention. At the completion of the reaction, each polynucleotide in the mixture is present in substantially equal concentration, regardless of the starting concentrations of the first and second amplicons. That is, the invention provides a method for synthesizing a normalized mixture of polynucleotides.

### FIELD OF THE INVENTION

The present invention relates to methods for synthesizing mixtures of nucleic acids, and more particularly, for synthesizing multiplexed nucleic acid probes.

### BACKGROUND

The use of complex mixtures of nucleic acid probes has increased as more and more large-scale genetic studies have taken place, which are designed to interrogate many thousands of genetic loci at the same time, Hardenbol et al, Nature Biotechnology, 21: 673-678 (2003; Fan et al, Genome Research, 10: 853-860 (2000); Chen et al, Genome Research, 10: 549-557 (2000); Hirschhorn et al, Proc. Natl. Acad. Sci., 97: 12164-12169 (2000); Lashkari et al, Proc. Natl. Acad. Sci., 94: 8945-8947 (1997). The production of complex mixtures of such probes can be expensive and labor-intensive if each probe is synthesized separately and then combined in the proper amounts for use. There have been attempts to address this problem by making use of oligonucleotides that are synthesized in parallel on microarrays, or like supports, e.g. Weiler et al, Anal. Biochem., 243: 218-227 (1996); Frank et al, Nucleic Acids Research, 11: 4365-4377 (1983); Lipschutz et al, U.S. Pat. No. 6,440,677. However, such approaches have not been practical for a variety of reasons, including poor and/or variable yields of individual species, unbalanced representation of the various sequences in a mixture, and difficulties in making sufficient quantities of polynucleotides for performing hybridization reactions.

The availability of methods of synthesizing mixtures of polynucleotides that overcome the deficiencies of prior art would greatly improve research, medical, and industrial applications that require large-scale multiplex or parallel analysis with hybridizations probes.

### SUMMARY OF THE INVENTION

The invention is directed to a method of convergently synthesizing mixtures of either single stranded or double stranded polynucleotides. In one aspect, oligonucleotides that form components of such polynucleotides are synthesized on one or more microarrays, or other large-scale parallel solid phase synthesis platforms, after which they are amplified directly, or are released into solution and then amplified. At least two sets of such released and amplified oligonucleotides are produced, referred to herein as first and second amplicons. The first and second amplicons are cleaved and then ligated to different ends of a bridging duplex that is present in the reaction in limiting quantity to form a polynucleotide mixture of the invention. At the completion of the reaction, each polynucleotide in the mixture is present in substantially equal concentration, regardless of the starting concentrations in the first and second amplicons. That is, the invention provides a method for synthesizing a normalized mixture of polynucleotides.

In another aspect, the invention provides a method of synthesizing a mixture of polynucleotide comprising the following steps: (a) amplifying a plurality of oligonucleotides from a first microarray to form a first amplicon, each oligonucleotide having a predetermined sequence comprising at least one first primer binding site at an end, a variable region, and a first cleavage site therebetween; (b) cleaving the first amplicon at the first cleavage site to form a first fragment having a first overhang with a nucleotide sequence, such that first fragments with different variable regions have first overhangs with different nucleotide sequences; (c) amplifying a plurality of oligonucleotides from a second microarray to form a second amplicon, each oligonucleotide having a predetermined sequence comprising at least one second primer binding site at an end, a variable region, and a second cleavage site therebetween; (d) cleaving the second amplicon at the second cleavage site to form a second overhang with a nucleotide sequence, such that second fragments with different variable regions have second overhangs with different nucleotide sequences; (e) ligating the first fragments and second fragments to a bridging duplex to form a mixture of polynucleotides, each bridging duplex having a first overhang and a second overhang such that ligation takes place if a first overhang of a first fragment is complementary with a first overhang of a bridging duplex and a second overhang of a second fragment is complementary with a second overhang of a bridging duplex.

In yet another aspect, the invention provides a method of synthesizing a mixture of polynucleotides comprising the following steps: (a) amplifying first and second oligonucleotides from one or more microarrays to form first and second amplicons, each first oligonucleotide having a predetermined sequence comprising at least one first primer binding site at an end, a variable region, and a first cleavage site therebetween and each second oligonucleotide having a predetermined sequence comprising at least one second primer binding site at an end, a variable region, and a second cleavage site therebetween; (b) cleaving the first and second amplicons at the first and second cleavage sites, respectively, to form first and second fragments with first and second overhangs, respectively, such that first fragments with different first overhangs have different variable regions and second fragments with different second overhangs have different variable regions; and (c) ligating the first fragments and second fragments to bridge duplexes to form a mixture of polynucleotides, each bridge oligonucleotides having a first overhang and a second overhang, such that ligation takes place if a first overhang of a first fragment is complementary with a first overhang of a bridging duplex and a second overhang of a second fragment is complementary with a second overhang of a bridging fragment.

In another aspect of the invention, in the step of ligating, the first fragments and the second fragments are in molar excess of the bridging duplexes so that substantially equimolar concentrations of polynucleotides are formed in the ligation reaction mixture.

The invention provides advances over prior approaches by providing normalized mixtures of polynucleotides assembled from component amplicons made from oligonucleotides efficiently synthesized on highly parallel synthesis platforms, such as microarrays, but which are of variable quality and concentration. Such polynucleotide mixtures are highly useful in constructing hybridization probes for large-scale genetic measurements.

### BRIEF DESCRIPTION OF THE FIGURES

FIGS. 1A-1C illustrate convergent assembly of first and second oligonucleotide mixtures with a bridging oligonucleotide to form a polynucleotide mixture of the invention.

FIG. 2 shows an application of polynucleotide mixtures of the invention for making molecular inversion probes.

### DEFINITIONS

Terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” in reference to tag complements means that the nucleotide sequence, or perhaps other physical or chemical characteristics, of an end-attached probe, such as a tag complement, can be determined from its address, i.e. a one-to-one correspondence between the sequence or other property of the end-attached probe and a spatial location on, or characteristic of, the solid phase support to which it is attached. Preferably, an address of a tag complement is a spatial location, e.g. the planar coordinates of a particular region containing copies of the end-attached probe. However, end-attached probes may be addressed in other ways too, e.g. by microparticle size, shape, color, frequency of micro-transponder, or the like, e.g. Chandler et al, PCT publication WO 97/14028.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g. conditions including tempature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-arninopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleoties or polynucleotides is not discernable, at least initially, from its location. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are descnbed in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Readout” means a characteristic of one or more signal generation moieties, or labels, that are measured, detected, and/or counted and that can be converted to a number or value. In one aspect, a readout of an assay is obtained by the use or application of a instrument and/or process that converts assay results on the molecular level into signals that may be detected and recorded. Such instrument or process may be referred to as a “readout device” (or instrument) or “readout process” (or method). A readout can also include, or refer to, an actual numerical representation of such collected or recorded data. For example, a readout of a hybridization assay using a microarray as a readout device collectively refers to signals generated at each feature, or hybridization site, of the microarray and their numerical, graphical, and/or pictorial representations.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecule in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

As used herein, the term “Tₘ” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation. Tm=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DETAILED DESCRIPTION OF THE INVENTION

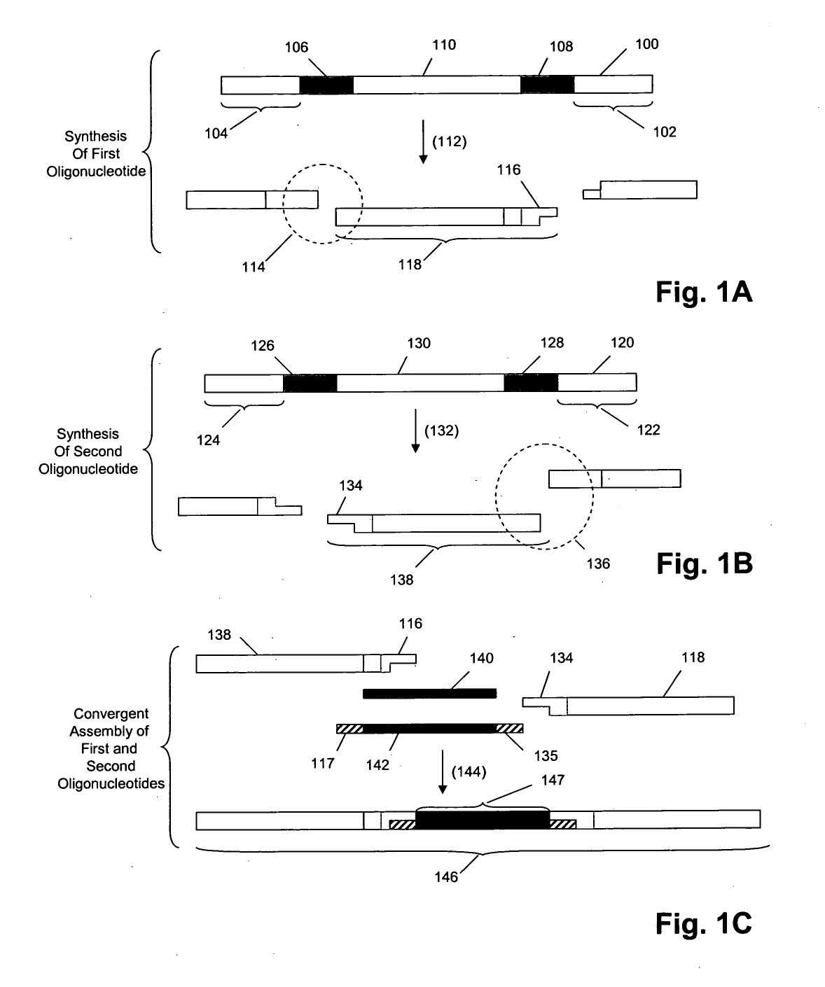

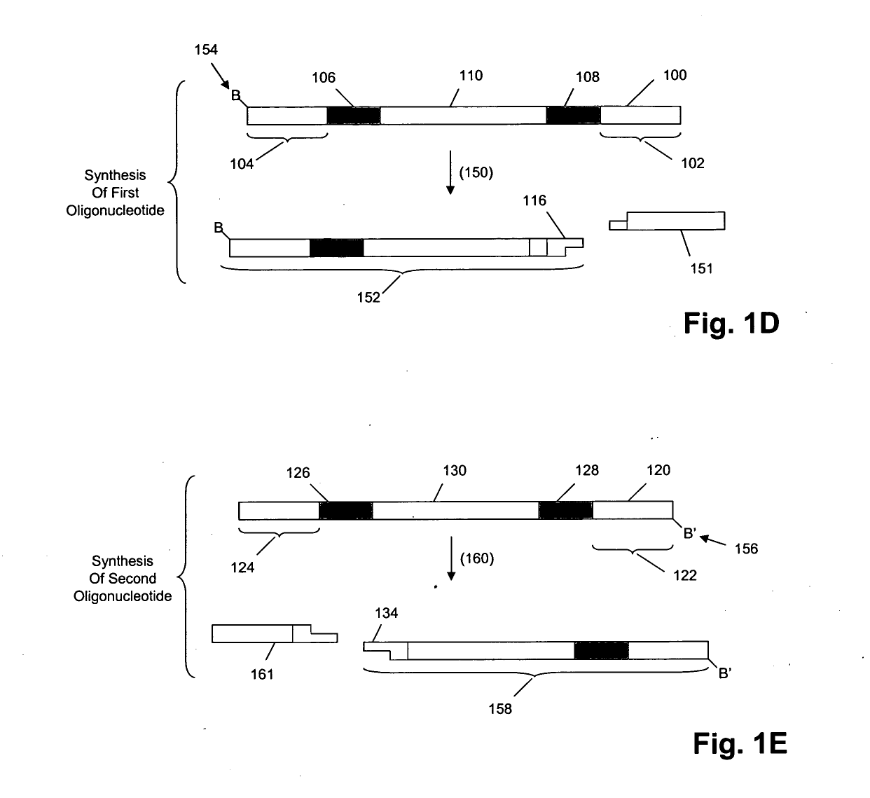

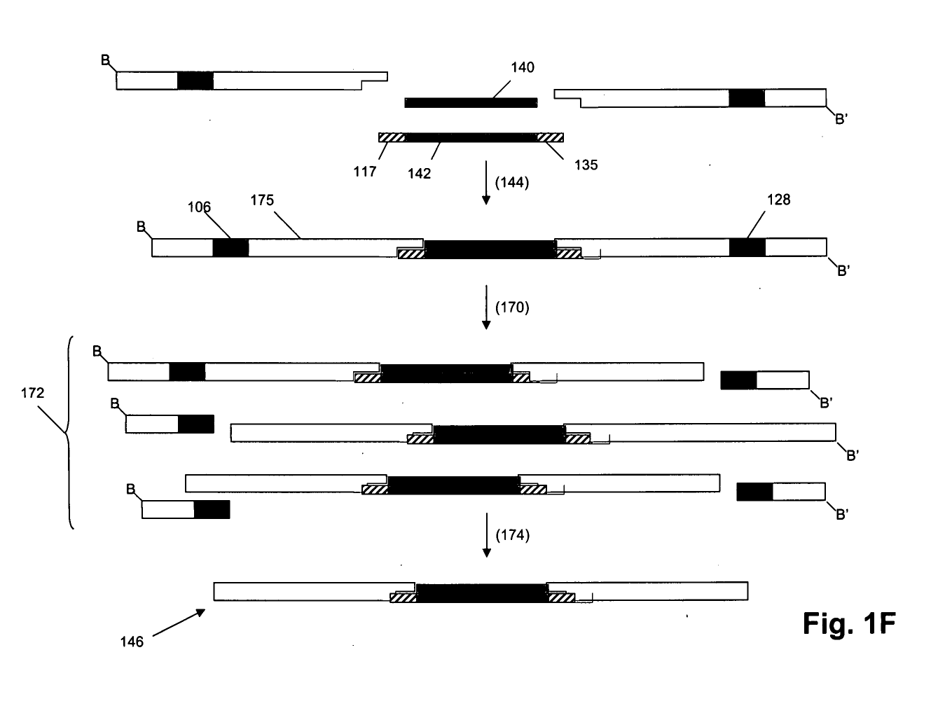

In one aspect, the invention provides an efficient and economical method for producing complex hybridization probes that may be employed in a variety of analytical techniques. FIGS. 1A-1C illustrate an exemplary embodiment of the invention that uses PCR to produce first and second amplicons. FIGS. 1A and 1B show elements of first and second amplicons that are produced from oligonucleotides that preferably are synthesized in parallel on a solid phase synthesis platform, such as one or more microarrays. The oligonucleotides from which first and second amplicons are made may be synthesized separately or together on the same one or more solid phase supports. The synthesis of high-density microarrays is disclosed in the following exemplary references that are incorporated by reference: Fodor et al, U.S. Pat. Nos. 5,424,186; 5,744,305; 5,445,934; 6,355,432; 6,440,667 (Affymetrix, Santa Clara, Calif.); Cerrina et al, U.S. Pat. No. 6,375,903 (NimbleGen, Madison, Wis.); and “ink-jet” synthesized microarrays, e.g. disclosed in Hughes et al, Nature Biotechnology, 19: 342-347 (2001); Caren et al U.S. Pat. No. 6,323,043 (Agilent Technologies, Palo Alto, Calif.); and the like. Preferably, a solid phase synthesis approach is selected that includes a capping step in each synthesis cycle, so that failure sequences are truncated. This is particularly advantageous when first and second amplicons are made by polymerase chain reactions, as only successfully completed sequences would have primer binding sites at both ends and thereby be amplified. The degree of amplification, e.g. the number of cycles if PCR is employed, depends on several factors including, but not limited to, the amount of product required, the complexity of the polynucleotide mixture, the length of the oligonucleotides from which first and second amplicons are made, and the like. For PCR amplifications, usually a conventional reaction of 25-30 cycles is performed in a reaction volume of 50-100 μL. Each mixture of first and second oligonucleotides contains pluralities of different oligonucleotides. In one aspect, such pluralities are limited only by the multiplexing capacity of solid phase synthesis. In another aspect, each such plurality is in the range of from 2 to 100,000; or in the range of from 2 to 50,000; or in the range of from 2 to 30,000; or in the range of from 2 to 20,000; or in the range of from 2 to 10,000; or in the range of from 2 to 5,000. The lengths of the oligonucleotide used to make the first and second amplicons may also vary widely. In one aspect, such lengths are limited only by the ability to produce sufficient starting material in the selected synthetic approach to permit subsequent amplification to the desired quantity for ligation. In another aspect, lengths of the oligonucleotides used to make the first and second amplicons may be selected in the range of from 18 to 150; or in the range of from 24 to 100; or in the range of from 24 to 75.

In one embodiment, as shown in FIG. 1A, the sequence of first amplicon (100) contains variable region (110) that successively is flanked, moving from the center towards the ends of the first amplicon, by cleavage sites (106) and (108), and by primer binding sites (102) and (104). Variable region (110) may contain one or more target-specific elements, such as a sequence complementary to a particular target nucleic acid that can serve as a specific hybridization probe, primer, or the like. Additionally, variable region (110) may contain an oligonucleotide tag for permitting the generation of a multiplexed assay readout, e.g. using an array of tag complements. Usually, within a mixture of first amplicons, different first amplicons have variable regions (110) with different sequences. In some embodiments, the sequences of all primer binding sites (104) may have the same sequence. Likewise, all primer binding sites (102) may have the same sequence. In such embodiments, the sequences of primer binding sites (102) and (104) may be the same or different. In other embodiments, there may be a plurality of subsets of first amplicons that have pairs of primer binding sites (102) and (104) such that each primer binding site (102) has the same sequence within a subset and each primer binding site (104) has the same sequence (the same or different from that of (102)) within the same subset. This permits a subset of first amplicons to be selectively amplified from a mixture if desired by using an appropriate pair of primers. After solid phase synthesis, first amplicon (100) is either directly amplified from its solid phase support, or it is first released from its solid phase support, and then amplified. A wide range of cleavable linkers may be employed if solution phase amplification is desired after synthesis, e.g. Weiler et al, Anal. Biochem., 243: 218-227 (1996); Letsinger et al, U.S. Pat. No. 5,112,962; Backes et al, Curr. Opin. Chem. Biol., 1: 86-93 (1997); and the like. In one aspect, first and second amplicons are amplified in a conventional amplification reaction, such as, a polymerase chain reaction (PCR), a NASBA reaction, or some variant thereof.

Cleavage site (106) is employed to remove primer binding site (104) prior to assembly of polynucleotide (146). The nature of cleavage site (106) is a routine design choice of one of ordinary skill in the art, wherein a primary consideration is the nature of the end desired on fragment (118). In one aspect, cleavage site (106) is a cleavage site for a commercially available restriction endonuclease. Preferably, such restriction endonuclease is a type us restriction endonuclease whose recognition site is located in primer binding site (104). The use of a type IIs restriction endonuclease permits the use of any sequence in cleavage site (106). Non-type IIs restriction endonucleases also can be used for cleavage at either site (106) or (108), with the understanding that the choice of the resulting end sequences are limited by the recognition sequences of the non-type IIs restriction endonucleases. Likewise to the above, cleavage site (108) is employed to remove primer binding site (102). However, it is also used to generate first overhang (116) having a predetermined sequence. As will be explained more filly below, the predetermined sequence of first overhang (116) is used to match fragment (118) with an appropriate fragment (138) by ligation to a common bridging duplex (147), made up of bridging oligonucleotides (140) and (142). First overhang (116) may be generated in a variety of ways, as explained more fully below. In one aspect, cleavage site (108) is cleaved with a type IIs restriction endonuclease having a recognition site in primer binding site (102).

After synthesis and amplification, first amplicon (100) is cleaved (112) at least at cleavage site (108), and optionally at cleavage site (106). In one aspect, fragment (118) is purified using a conventional technique, e.g. preparative gel electrophoresis, or the like, before combining with bridging oligonucleotide (142) and its complement (140) in a ligation reaction. In other aspects, a crude reaction mixture containing fragment (118) may be used directly, provided that a polymerase is selected that does not destroyed first overhang (116), or alternatively, the polymerase is inactivated after amplification.

The preparation of second amplicon (120) proceeds similarly to the above. As shown in FIG. 1B, the sequence of second amplicon (120) contains variable region (130) that successively is flanked, moving from the center towards the ends of the second amplicon, by cleavage sites (126) and (128), and by primer binding sites (122) and (124). As with the variable region of the first amplicon, variable region (130) may contain one or more target-specific elements, such as a sequence complementary to a particular target nucleic acid that can serve as a specific hybridization probe, primer, or the like, as well as an oligonucleotide tag or other elements, e.g. restriction sites, primer binding sites, and the like, associated with a multiplexed readout.

As with first amplicons, within a mixture of second amplicons, different second amplicons have variable regions (130) with different sequences. In some embodiments, the sequences of all primer binding sites (124) may have the same sequence and all primer binding sites (122) may have the same sequence. In other embodiments, there may be a plurality of subsets of second amplicons that have pairs of primer binding sites (122) and (124) that have the same sequences within a subset, but different sequences as between different subsets, so that a subset of second amplicons may be selectively amplified from a mixture if desired. After solid phase synthesis, second amplicon (120) is either directly amplified from its solid phase support, or it is first released from its solid phase support, and then amplified. As with the first amplicon, a wide range of cleavable linkers may be employed if solution phase amplification is desired after synthesis, as described above. Usually, within a mixture of second amplicons, different second amplicons have variable regions (120) with different sequences. Cleavage site (128) is employed to remove primer binding site (122) prior to assembly of polynucleotide (146). The nature of cleavage site (128) is a routine design choice of one of ordinary skill in the art, wherein a primary consideration is the nature of the end desired on fragment (138). In one aspect, cleavage site (128) is a cleavage site for a commercially available restriction endonuclease. Preferably, such restriction endonuclease is a type IIs restriction endonuclease whose recognition site is located in primer binding site (122). The use of a type IIs restriction endonuclease permits the use of any sequence in cleavage site (128). Cleavage site (126) is employed to remove primer binding site (124) and to generate second overhang (134) having a predetermined sequence. Similarly to first overhang (116), the predetermined sequence of second overhang (134) is used to match fragment (138) with an appropriate fragment (118) by ligation to a common bridging duplex (147) having complementary overhangs. In one aspect, cleavage site (126) is cleaved with a type IIs restriction endonuclease having a recognition site in primer binding site (124). After synthesis and amplification, second amplicon (120) is cleaved (132) at least at cleavage site (126), and optionally at cleavage site (128). Preferably, fragment (138) is purified using a conventional technique, e.g. preparative gel electrophoresis, or the like, before combining with bridging duplex (142) and its complement (140) in a ligation reaction. Alternatively, as described above, fragment (138) may used directly from a cleavage reaction mixture.

After fragments (118) and (138) are prepared, and optionally purified, they are combined with bridging oligonucleotide (142) and its complement (140) in a ligation reaction (144) that results in polynucleotide (146), as illustrated in FIG. 1C. Reaction conditions are selected so that the amount of polynucleotide (146) formed is controlled by the concentration selected for bridging oligonucleotide (142). In one aspect, bridging oligonucleotide (142) is present in limiting concentration (or amounts) in reaction (144), so that (ideally) when the reaction is completed, all of bridging oligonucleotide (142) will be incorporated into product (146) and, at the same time, there will be some non-zero concentration of each of fragments (118) and (138), and optionally (140) left over. Preferably, bridging oligonucleotide (140) is present in equal or greater concentration than that of bridging oligonucleotide (142). In another aspect, the concentration of each species of fragments (118) and (138) is selected so that each is in substantial molar excess of its associated bridging duplex (142). In one aspect, substantial molar excess means that each species of fragments (118) and (138) is in the range of from 2 to 100 times the concentration of its associated bridging duplex (142). In another aspect, each species of fragments (118) and (138) is in the range of from 5 to 10 times the concentration of its associated bridging oligonucleotide (142). Bridging oligonucleotides (142) and their complements (140) are synthesized using a conventional commercially available technique, e.g. solid phase synthesis using phosphoramidite chemistry, followed by purification, e.g. by HPLC. Preferably, each bridging oligonucleotide (142) is used in ligation reaction (144) in substantially the same quantities, or concentrations, so that substantially equivalent quantities of the resulting polynucleotides (146) are produced. Preferably, the concentrations of the different species of polynucleotide (146) at the completion of reaction (144) differ by no more than ten fold, and more preferably, by no more than five fold, and still more preferably, by no more than three fold.

The sequences of overhangs (117) and (135) of bridging oligoncleotide (142) are used to pair fragments (118) and (138) of first and second amplicons, respectively, in a predetermined manner. (In fact, the sequences and types of overhangs (that is, whether the 3′ end or 5′ end is recessive) can be viewed as encoding the variable region.) For example, in the construction of molecular inversion probes, as described below, variable regions in a pair of first and second amplicons may correspond to well-defined adjacent hybridization sites on a target nucleic acid. For such a probe to work properly, fragments (118) and (138) from such first and second amplicons must be paired together correctly. This is accomplished by establishing a correspondence between the sequence of a first overhang and the identify of its corresponding variable region (110), so that whenever such sequences are in the same ligation reaction, each different variable region (110) is linked to a different first overhang. Likewise, a similar correspondence is established between the sequence of a second overhang and the identity of its corresponding variable region (130). Consequently, selected variable regions (110) and (130) are linked together by being ligated to an appropriate bridging duplex (142) that has ends (117) and (135) complementary with first overhang (116) and (134), respectively. As mentioned above, in one aspect of the invention, the various overhangs may be generated using a type IIs restriction endonuclease. Since the larger the overhang, the more flexibility in combining different fragments, preferably, a type IIs restriction endonuclease is used that generates the largest overhang as possible. Thus, in one aspect, first and second overhangs are generated by a type IIs restriction endonuclease that leaves a four-nucleotide overhang. Exemplary type IIs restriction endonucleases that generate four-nucleotide or greater overhangs include Bst XI, Aar I, Bfu AI, Bsm AI, Bsm BI, Bsm FI, Bsp MI, Fok I, Hga I, and the like (available from New England Biolabs, Berverly, Mass.). For longer overhangs, other techniques may be used including a “stripping” reaction using a T4 DNA polymerase as disclosed in Brenner et al, Proc. Natl. Acad. Sci., 97: 1665-1670 (2000); or by incorporating dUTPs that are removed by treating with a uracil-DNA glycosylase and/or heat to form ends of greater than four nucleotides, e.g. as disclosed by Rombel et al, Biotechniques, 34: 244-250 (2003).

In another aspect, first and second overhangs (116) and (134) are of opposite type to permit the maximum number of different fragments (118) and (138) to be joined. For example, as shown in FIG. 1C, first overhang (116) is a 3′-protruding overhang (or equivalently a 5″-recessed overhang) and second overhang (134) is a 5′-protruding overhang (or equivalently a 3′-recessed overhang). Thus, in the ligation reaction, spurious joining of fragment (118) to fragment (138) is precluded. This aspect permits the highest degree of multiplexing in the ligation reaction when four-base overhangs are employed. In other embodiments, fragments (118) and (138) with compatible overhangs (e.g., 3′-protruding and 3′-recessed, respectively, or 5′-protruding and 5′-recessed, respectively) may be employed; however, the sequences of first and second overhangs (116) and (134) must be selected so that no first overhang sequence is complementary with any second overhang sequence. Thus, even under ideal circumstances, a multiplex level of only 128 can be achieved in a ligation reaction when four-nucleotide overhangs are available.

The terminal nucleotides of fragment (118), bridging oligonucleotide (140), and bridging oligonucleotide (142) may be selectively phosphorylated to control whether a double stranded product or a single stranded product is formed. That is, both stands of each fragment (118) and (138) may be ligated to bridging duplex (147), or the 5′ ends of selected strands may be left unphosphorylated so that no ligation takes place. In one aspect of the invention, a single stranded polynucleotide is desired, for example, the upper stand of product (146). In this embodiment, the 5′ end of bridging oligonucleotide, and the 5′ protruding stand of fragment (118) are phosphorylated prior to including them in the ligation reaction. Such 5′ phosphate groups may be added enzymatically using a conventional kinase reaction or chemically using a commercially available phosphorylating agent (e.g. Glen Research, Sterling, Va.). Whenever a single stranded product is desired, e.g. comprising the upper strand of product (146), it may readily be isolated from the reaction mixture by conventional separation techniques, e.g. preparative gel electrophoresis.

The degree of multiplexing that can be achieved by the invention may be controlled by the use of different pairs of primers (for selectively amplifying first and second amplicons) and different lengths of first and second overhangs. In one aspect, first and second overhangs are each four nucleotides, thereby permitting up to 256 (=4⁴) fragments (118) to be linked with up to 256 fragments (138). That is, up to 256×256=65,536 polynucleotides may be synthesized in a ligation reaction. In another aspect of the invention, first subsets of first amplicons are produced and second subsets of second amplicons are produced, where each subset is defined by different pairs of primers.

Another embodiment of the present invention is illustrated in FIGS. 1D-1F. This embodiment is similar to that described in FIGS. 1A-1C, except that restriction endonuclease sites (106) and (128) are cleaved later in the process, and fragments (110) and (130) are labeled with one or more capture moieties, such as (154) and (156), respectively, e.g. during PCR amplification. In some versions, amplicons (110) and (130) can have two different capture moieties attached to opposite ends, so that during or after cleavage step (150) or (160) fragments (151) and (161), respectively, can be removed from the reaction mixture by a complementary capture agent. Otherwise, fragments (152) and (158) are prepared as described above. Capture moieties B and B′ can be haptens, such as biotin, desthiobiotin, digoxigenin, fluorescein, dinitrophenol, or the like. Corresponding capture agents can be antidodies specific for the haptens, or in the case of biotin, streptavidin or avidin. This embodiment provides an alternative pathway for convergent assembly of polynucleotide (146) in which incomplete digestion products from cleavage steps (150) or (160) can be easily eliminated from the reaction mixture. Thus, when assembled polynucleotide (175) is digested (170) at restriction sites (106) and (128) to give possible products (172), from which desired polynucleotide (146) can be eluted (174) after treatment with the respective complementary capture agents of B and B′.

In one aspect, polynucleotide mixtures of the invention may be employed as circularizing probes, such as padlock probes, rolling circle probes, molecular inversion probes, linear amplification molecules for multiplexed PCR, and the like, e.g. padlock probes being disclosed in U.S. Pat. Nos. 5,871,921; 6,235,472; 5,866,337; and Japanese patent JP 4-262799; rolling circle probes being disclosed in Aono et al, JP4-262799; Lizardi, U.S. Pat. Nos. 5,854,033; 6,183,960; 6,344,239; molecular inversion probes being disclosed in Hardenbol et al (cited above) and in Willis et al, U.S. patent publication 2004/0101835; and linear amplification molecules being disclosed in Faham et al, U.S. patent publication 2003/0104459; all of which are incorporated herein by reference. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatatemers, and the like, with an exonuclease, such as exonuclease I.

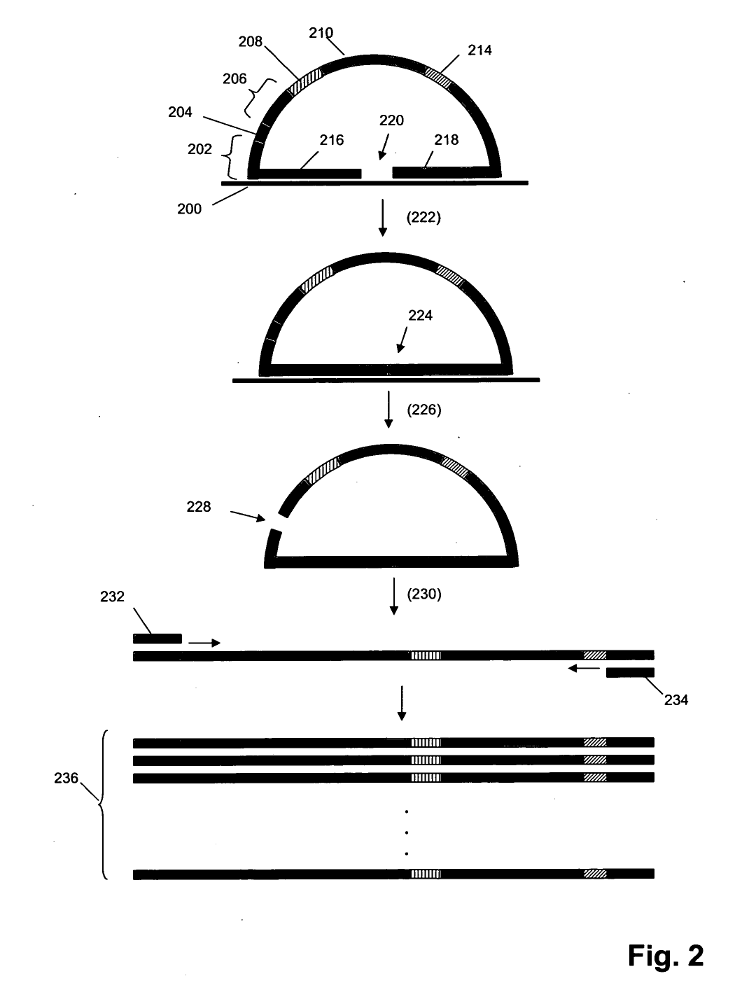

FIG. 2 illustrates a molecular inversion probe and how it can be used to generate an amplicon after interacting with a target polynucleotide in a sample. A linear version of the probe is combined with a sample containing target polynucleotide (200) under conditions that permit target-specific region 1 (216) and target-specific region 2 (218) to form stable duplexes with complementary regions of target polynucleotide (200). The ends of the target-specific regions may abut one another (being separated by a “nick”) or there may be a gap (220) of several (e.g. 1-10 nucleotides) between them. In either case, after hybridization of the target-specific regions, the ends of the two target specific regions are covalently linked by way of a ligation reaction or an extension reaction followed by a ligation reaction, i.e. a so-called “gap-ligation” reaction. The latter reaction is carried out by extending with a DNA polymerase a free 3′ end of one of the target-specific regions so that the extended end abuts the end of the other target-specific region, which has a 5′ phosphate, or like group, to permit ligation. In one aspect, a molecular inversion probe has a structure as illustrated in FIG. 2. Besides target-specific regions (216 and 218), in sequence such a probe may include first primer binding site (202), cleavage site (204), second primer binding site (206), first tag-adjacent sequences (208) (usually restriction endonuclease sites and/or primer binding sites) for tailoring one end of a labeled target sequence containing oligonucleotide tag (210), and second tag-adjacent sequences (214) for tailoring the other end of a labeled target sequence. Alternatively, cleavage-site (204) may be added at a later step by amplification using a primer containing such a cleavage site. In operation, after specific hybridization of the target-specific regions and their ligation (222), the reaction mixture is treated with a single stranded exonuclease that preferentially digests all single stranded nucleic acids, except circularized probes. After such treatment, circularized probes are treated (226) with a cleaving agent that cleaves the probe between primer (202) and primer (206) so that the structure is linearized (230). Cleavage site (204) and its corresponding cleaving agent is a design choice for one of ordinary skill in the art. In one aspect, cleavage site (204) is a segment containing a sequence of uracil-containing nucleotides and the cleavage agent is treatment with uracil-DNA glycosylase followed by heating. After the circularized probes are opened, the linear product is amplified, e.g. by PCR using primers (232) and (234), to form amplicons (236). A multiplexed readout may be obtained from amplicon (236) by labeling and excising oligonucleotide tag (210) and specifically hybridizing the labeled tags to a microarray of tag complements, e.g. a GenFlex array (Affymetrix, Santa Clara, Calif.); a bead array (Illumina, San Diego, Calif.); or a fluid array, e.g. Chandler et al, U.S. Pat. No. 5,981,180 (Lumenix, Austin, Tex.).

### Oligonucleotide Tags and Minimally Cross-Hybridizing Sets

In one aspect, the invention provides end-attached probes and labeled target sequences that comprise minimally cross-hybridizing sets of oligonucleotide tags, such as disclosed in Brenner et al, U.S. Pat. No. 5,846,719; Mao et al (cited above); Fan et al, International patent publication WO 2000/058516; Morris et al, U.S. Pat. No. 6,458,530; Morris et al, U.S. patent publication 2003/0104436; Church et al, European patent publication 0 303 459; Huang et al, U.S. Pat. No. 6,709,816; which references are incorporated herein by reference. The sequences of oligonucleotides of a minimally cross-hybridizing set differ from the sequences of every other member of the same set by at least two nucleotides, and more preferably, by at least three nucleotides. Thus, each member of such a set cannot form a duplex (or triplex) with the complement of any other member with less than two mismatches, or three mismatches as the case may be. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleotide tags. In particular, tag complements may comprise peptide nucleic acids (PNAs). Oligonucleotide tags from the same minimally cross-hybridizing set when used with their corresponding tag complements provide a means of enhancing specificity of hybridization. Microarrays of tag complements are available commercially, e.g. GenFlex Tag Array (Affymetrix, Santa Clara, Calif.); and their construction and use are disclosed in Fan et al, International patent publication WO 2000/058516; Morris et al, U.S. Pat. No. 6,458,530; Morris et al, U.S. patent publication 2003/0104436; and Huang et al (cited above).

As mentioned above, in one aspect tag complements comprise PNAs, which may be synthesized using methods disclosed in the art, such as Nielsen and Egholm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. No. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference. Construction and use of microarrays comprising PNA tag complements are disclosed in Brandt et al, Nucleic Acids Research, 31(19), e119 (2003).

Preferably, oligonucleotide tags and tag complements are selected to have similar duplex or triplex stabilities to one another so that perfectly matched hybrids have similar or substantially identical melting temperatures. This permits mis-matched tag complements to be more readily distinguished from perfectly matched tag complements in the hybridization steps, e.g. by washing under stringent conditions. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Nail. Acad. Sci., 83: 3746-3750 (1986); Wetmur, Crit. Rev. Biochem. Mol. Biol., 26: 227-259 (1991); and the like. A minimally cross-hybridizing set of oligonucleotides may be screened by additional criteria, such as GC-content, distribution of mismatches, theoretical melting temperature, and the like, to form a subset which is also a minimally cross-hybridizing set.

### Hybridization of Labeled Target Sequence to Solid Phase Supports

Methods for hybridizing labeled target sequences to microarrays, and like platforms, suitable for the present invention are well known in the art. Guidance for selecting conditions and materials for applying labeled target sequences to solid phase supports, such as microarrays, may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about 1 M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to no more than 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled target sequence to a GenFlex™ microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂, PO₄, 6 mM EDTA (pH 7.4), 0.005% Triton X-100)+0.5 mg/ml of BSA for a few minutes, then hybridized with 120 μL hybridization solution (as described below) at 42° C. for 2 hours on a rotisserie, at 40 RPM. Hybridization Solution consists of 3M TMACL (Tetramethylammonium. Chloride), 50 mM MES ((2-[N-Morpholino]ethanesulfonic acid) Sodium Salt) (pH 6.7), 0.01% of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie, at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commnercially from Affymetrix) with a resolution of 60-70 pixels per feature and filters and other settings as appropriate for the labels employed. GeneChip Software (Affymetrix) may be used to convert the image files into digitized files for further data analysis.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

We claim:

1. A method of synthesizing a mixture of polynucleotides, the method comprising the steps of:
(a) amplifying a plurality of oligonucleotides from a first microarray to form a first amplicon, each oligonucleotide having a predetermined sequence comprising at least one first primer binding site at an end, a variable region, and a first cleavage site therebetween;
(b) cleaving the first amplicon at the first cleavage site to form a first fragment having a first overhang with a nucleotide sequence, such that first fragments with different variable regions have first overhangs with different nucleotide sequences;
(c) amplifying a plurality of oligonucleotides from a second microarray to form a second amplicon, each oligonucleotide having a predetermined sequence comprising at least one second primer binding site at an end, a variable region, and a second cleavage site therebetween;
(d) cleaving the second amplicon at the second cleavage site to form a second overhang with a nucleotide sequence, such that second fragments with different variable regions have second overhangs with different nucleotide sequences;
(e) ligating the first fragments and second fragments to a bridging duplex to form a mixture of polynucleotides, each bridging duplex having a first overhang and a second overhang such that ligation takes place if a first overhang of a first-fragment is complementary a first overhang of a bridging duplex and a second overhang of a second fragment is complementary with a second overhang of a bridging duplex.

2. The method of claim 1 wherein said first and second cleavage sites are each restriction sites and wherein said step of cleaving includes treating said first amplicon and said second amplicon with a restriction endonuclease.

3. The method of claim 2 wherein said first overhang is a 3′-protruding overhang and said second overhang is a 5′-protruding overhang.

4. A method of synthesizing a mixture of polynucleotides, the method comprising the steps of:
(a) amplifying first and second oligonucleotides from one or more microarrays to form first and second amplicons, each first oligonucleotide having a predetermined sequence comprising at least one first primer binding site at an end, a variable region, and a first cleavage site therebetween and each second oligonucleotide having a predetermined sequence comprising at least one second primer binding site at an end, a variable region, and a second cleavage site therebetween;
(b) cleaving the first and second amplicons at the first and second cleavage sites, respectively, to form first and second fragments with first and second overhangs, respectively, such that first fragments with different first overhangs have different variable regions and second fragments with different second overhangs have different variable regions; and
(c) ligating the first fragments and second fragments to bridge duplexes to form a mixture of polynucleotides, each bridge oligonucleotides having a first overhang and a second overhang, such that ligation takes place if a first overhang of a first fragment is complementary with a first overhang of a bridging duplex and a second overhang of a second fragment is complementary with a second overhang of a bridging fragment.

5. The method of claim 4 wherein in said step of ligating said first fragments and said second fragments are in molar excess of said bridging duplexes.
